(19) United States
(12) Patent Application Publication
(10) Pub. No.: US 2006/0025668 A1
(43) Pub. Date: Feb. 2, 2006

(54) Operating table with embedded tracking technology

(76) Inventors: Thomas H. Peterson, Wilmington, MA (US); Lewis Levine, Weston, MA (US); Peter Traneus Anderson, Andover, MA (US)

Correspondence Address: MCANDREWS HELD & MALLOY, LTD, 500 WEST MADISON STREET, SUITE 3400, CHICAGO, IL, 60661, US

(21) Appl. No.: 10/909,461

(22) Filed: Aug. 2, 2004

(51) Int. Cl. A61B 5/05

(52) U.S. Cl. 600/407

(57) ABSTRACT

A system and method for embedding tracking technology in a medical table is disclosed. A plurality of table sensors is attached to a medical table to form an array. An instrument sensor is attached to an instrument. At least one of the table sensors and the instrument sensor generates at least one magnetic dipole field. The instrument sensor is moved relative to the array while at least one of the table sensors and the instrument sensor measures at least one vector component of the field. The measured vector component(s) are communicated to tracker electronics that determine at least one of a position and orientation of the instrument sensor relative to the array.

### BACKGROUND OF THE INVENTION

The present invention generally relates to an electromagnetic tracking system. In particular, the present invention relates to a system and method for embedding tracking technology in a medical table.

Medical practitioners, such as doctors, surgeons, and other medical professionals, often rely upon technology when performing a medical procedure, such as image-guided surgery or examination. A tracking system may provide positioning information for the medical instrument with respect to the patient or a reference coordinate system, for example. A medical practitioner may refer to the tracking system to ascertain the position of the medical instrument when the instrument is not within the practitioner's line of sight. A tracking system may also aid in pre-surgical planning.

The tracking or navigation system allows the medical practitioner to visualize the patient's anatomy and track the position and orientation of the instrument. The medical practitioner may use the tracking system to determine when the instrument is positioned in a desired location. The medical practitioner may locate and operate on a desired or injured area while avoiding other structures. Increased precision in locating medical instruments within a patient may provide for a less invasive medical procedure by facilitating improved control over smaller instruments having less impact on the patient. Improved control and precision with smaller, more refined instruments may also reduce risks associated with more invasive procedures such as open surgery.

Tracking systems may also be used to track the position of items other than medical instruments in a variety of applications. That is, a tracking system may be used in other settings where the position of an instrument in an object or an environment is difficult to accurately determine by visual inspection. For example, tracking technology may be used in forensic or security applications. Retail stores may use tracking technology to prevent theft of merchandise. In such cases, a passive transponder may be located on the merchandise. A transmitter may be strategically located within the retail facility. The transmitter emits an excitation signal at a frequency that is designed to produce a response from a transponder. When merchandise carrying a transponder is located within the transmission range of the transmitter, the transponder produces a response signal that is detected by a receiver. The receiver then determines the location of the transponder based upon characteristics of the response signal.

Tracking systems are also often used in virtual reality systems or simulators. Tracking systems may be used to monitor the position of a person in a simulated environment. A transponder or transponders may be located on a person or object. A transmitter emits an excitation signal and a transponder produces a response signal. A receiver detects the response signal. The signal emitted by the transponder may then be used to monitor the position of a person or object in a simulated environment.

Tracking systems may be ultrasound, inertial position, or electromagnetic tracking systems, for example. Electromagnetic tracking systems may employ coils as receivers and transmitters. Typically, an electromagnetic tracking system is configured in an industry-standard coil architecture (“ISCA”). ISCA uses three collocated orthogonal quasi-dipole transmitter coils and three collocated quasi-dipole receiver coils. Other systems may use three large, non-dipole, non-collocated transmitter coils with three collocated quasi-dipole receiver coils. Another tracking system architecture uses an array of six or more transmitter coils spread out in space and one or more quasi-dipole receiver coils. Alternatively, a single quasi-dipole transmitter coil may be used with an array of six or more receivers spread out in space.

The ISCA tracker architecture uses a three-axis dipole coil transmitter and a three-axis dipole coil receiver. Each three-axis transmitter or receiver is built so that the three coils exhibit the same effective area, are oriented orthogonal to one another, and are centered at the same point. If the coils are small enough compared to a distance between the transmitter and receiver, then the coil may exhibit dipole behavior. The trio of receiver coils may detect magnetic fields generated by the trio of transmitter coils. Using three approximately concentrically positioned transmitter coils and three approximately concentrically positioned receiver coils, for example, nine parameter measurements may be obtained. From the nine parameter measurements a position and orientation calculation may determine position and orientation information for each of the transmitter coils with respect to the receiver coil trio with three degrees of freedom.

In medical and surgical imaging, such as intraoperative or perioperative imaging, images are formed of a region of a patient's body. The images are used to aid in an ongoing procedure with a surgical tool or instrument applied to the patient and tracked in relation to a reference coordinate system formed from the images. Image-guided surgery is of a special utility in surgical procedures such as brain surgery and arthroscopic procedures on the knee, wrist, shoulder or spine, as well as certain types of angiography, cardiac procedures, interventional radiology and biopsies in which x-ray images may be taken to display, correct the position of, or otherwise navigate a tool or instrument involved in the procedure.

Several areas of surgery involve very precise planning and control for placement of an elongated probe or other article in tissue or bone that is internal or difficult to view directly. In particular, for brain surgery, stereotactic frames that define an entry point, probe angle and probe depth are used to access a site in the brain, generally in conjunction with previously compiled three-dimensional diagnostic images, such as MRI, PET or CT scan images, which provide accurate tissue images. For placement of pedicle screws in the spine, where visual and fluoroscopic imaging directions may not capture an axial view to center a profile of an insertion path in bone, such systems have also been useful.

When used with existing CT, PET or MRI image sets, previously recorded diagnostic image sets define a three dimensional rectilinear coordinate system, either by virtue of their precision scan formation or by the spatial mathematics of their reconstruction algorithms. However, it may be desirable to correlate the available fluoroscopic views and anatomical features visible from the surface or in fluoroscopic images with features in the 3-D diagnostic images and with external coordinates of tools being employed. Correlation is often done by providing implanted fiducials and adding externally visible or trackable markers that may be imaged. Using a keyboard or mouse, fiducials may be identified in the various images. Thus, common sets of coordinate registration points may be identified in the different images. The common sets of coordinate registration points may also be trackable in an automated way by an external coordinate measurement device, such as a suitably programmed off-the-shelf optical tracking assembly. Instead of imageable fiducials, which may for example be imaged in both fluoroscopic and MRI or CT images, such systems may also operate to a large extent with simple optical tracking of the surgical tool and may employ an initialization protocol wherein a surgeon touches or points at a number of bony prominences or other recognizable anatomic features in order to define external coordinates in relation to a patient anatomy and to initiate software tracking of the anatomic features.

Generally, image-guided surgery systems operate with an image display which is positioned in a surgeon's field of view and which displays a few panels such as a selected MRI image and several x-ray or fluoroscopic views taken from different angles. Three-dimensional diagnostic images typically have a spatial resolution that is both rectilinear and accurate to within a very small tolerance, such as to within one millimeter or less. By contrast, fluoroscopic views may be distorted. The fluoroscopic views are shadowgraphic in that they represent the density of all tissue through which the conical x-ray beam has passed. In tool navigation systems, the display visible to the surgeon may show an image of a surgical tool, biopsy instrument, pedicle screw, probe or other device projected onto a fluoroscopic image, so that the surgeon may visualize the orientation of the surgical instrument in relation to the imaged patient anatomy. An appropriate reconstructed CT or MRI image, which may correspond to the tracked coordinates of the probe tip, may also be displayed.

Among the systems that have been proposed for effecting such displays, many rely on closely tracking the position and orientation of the surgical instrument in external coordinates. The various sets of coordinates may be defined by robotic mechanical links and encoders, or more usually, are defined by a fixed patient support, two or more receivers such as video cameras which may be fixed to the support, and a plurality of signaling elements attached to a guide or frame on the surgical instrument that enable the position and orientation of the tool with respect to the patient support and camera frame to be automatically determined by triangulation, so that various transformations between respective coordinates may be computed. Three-dimensional tracking systems employing two video cameras and a plurality of emitters or other position signaling elements have long been commercially available and are readily adapted to such operating room systems. Similar systems may also determine external position coordinates using commercially available acoustic ranging systems in which three or more acoustic emitters are actuated and their sounds detected at plural receivers to determine their relative distances from the detecting assemblies, and thus define by simple triangulation the position and orientation of the frames or supports on which the emitters are mounted. When tracked fiducials appear in the diagnostic images, it is possible to define a transformation between operating room coordinates and the coordinates of the image.

In general, the feasibility or utility of a system of this type depends on a number of factors such as cost, accuracy, dependability, ease of use, speed of operation and the like. Current tracking systems typically involve many pieces of hardware to perform image-guided surgery and other imaging or surgical operations. Because of the many required separate components, physical interference commonly occurs between the patient, the surgeon, the medical instrument or device, the operating or imaging table and/or the various tracking sensors.

Thus, a need exists for a system and method for embedded tracking technology in a medical table. Such a system and method can provide for the reduction of the amount of components required for a tracking system in an operating and/or imaging room environment.

Also, by embedding tracking technology into a table, a reduction in magnetic field distortions can be achieved. For example, by embedding tracking technology into a table, a coil array can become fixed with respect to the table. In so fixing a coil array, magnetic field distortions normally caused by a table may be corrected by creating a magnetic field map at the time the table is manufactured. In contrast, by not embedding the tracking technology, any field distortion caused by the table must either be accounted for by creating a distortion-free table or by mapping the magnetic field before each and every use.

### BRIEF SUMMARY OF THE INVENTION

The present invention provides a medical table system with embedded tracking technology. The system includes a plurality of table sensors, an instrument sensor, at least one magnetic dipole field, at least one vector component of the field, and tracker electronics. The table sensors are inserted in the table to create a table sensor array. The instrument sensor is moved relative to the array. The magnetic dipole field is generated by at least one of the table sensors and the instrument sensor. The vector component of the field is measured by at least one of the table sensors and the instrument sensor. The tracker electronics determine at least one of a position and an orientation of the instrument sensor relative to the array using the vector component.

The present invention also provides a method for embedding tracking technology in a medical table. The method includes inserting a plurality of table sensors in the table to create a table sensor array, generating at least one magnetic dipole field, moving an instrument sensor relative to the array, measuring at least one vector component of the field, and determining at least one of a position and an orientation of the instrument sensor relative to the array using the vector component.

The present invention also provides for a wireless embedded tracking system. The system includes a wireless sensor transmitting at least one magnetic dipole field, an array of receiving sensors measuring the at least one field, a medical table embedded with the array, where the wireless sensor is moved relative to the table, and tracking electronics determining at least one of a position and an orientation of the wireless sensor relative to the array.

### BRIEF DESCRIPTION OF SEVERAL VIEWS OF THE DRAWINGS

FIG. 1 illustrates a medical table system with embedded tracking technology used in accordance with an embodiment of the present invention.

FIG. 2 illustrates a medical table used in accordance with an embodiment of the present invention.

FIG. 3 illustrates a retrofitted medical table used in accordance with an embodiment of the present invention.

FIG. 4 illustrates a flow diagram for a method for embedding tracking technology in a medical table used in accordance with an embodiment of the present invention.

### DETAILED DESCRIPTION OF THE INVENTION

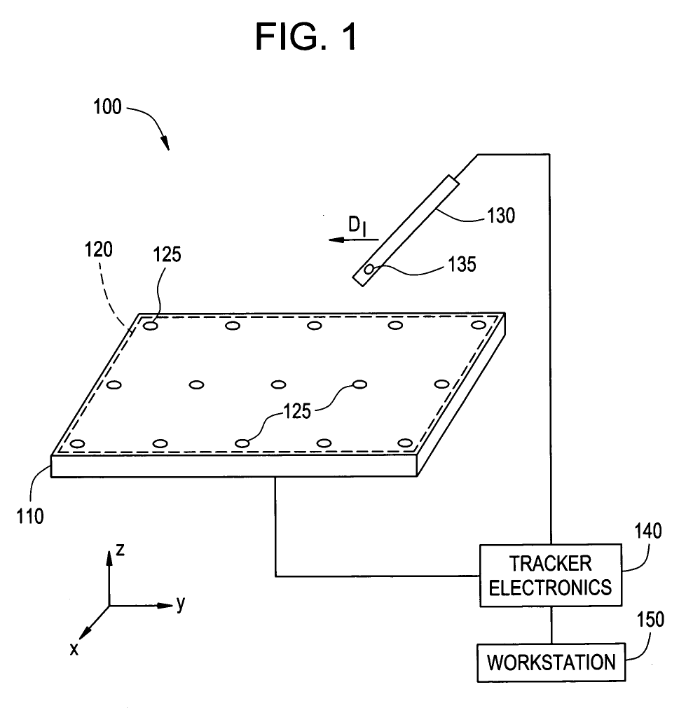

FIG. 1 illustrates a medical table system 100 with embedded tracking technology used in accordance with an embodiment of the present invention. System 100 includes a medical table 110, a table sensor array 120, a medical instrument 130, tracker electronics 140 and workstation 150. Table 110 may include, for example, an operating room table, an x-ray imaging table, a combination operating and imaging table, or a Jackson table, generally used for spine and orthopedic applications. In addition, table 110 can include any other medical apparatus that could benefit from tracking technology, including, for example, a C-arm useful in x-ray examinations of patients. Table sensor array 120 includes a plurality of table sensors 125. Medical instrument 130 includes an instrument sensor 135. Workstation 150 may include a memory and input and output devices (not shown).

In the table system 100, table sensors 125 are inserted in table 110. Table sensors 125 can be inserted by removably or permanently attaching each table sensor 125 to a surface of table 110. Table sensors 125 can be inserted in a regular grid throughout table 110, for example, as shown in FIG. 1. Instrument sensor 135 can be attached to medical instrument 130. Instrument sensor 135 may be removably or permanently attached to medical instrument 130. At least one of table sensors 125 and instrument sensor 135 can be connected to tracker electronics 140 so as to enable communication between at least one of table sensors 125 and/or instrument sensor 135 and tracker electronics 140. Table sensors 125 and/or instrument sensor 135 can be connected to tracker electronics 140 by a wired or wireless electrical connection. Tracker electronics 140 are connected to workstation 150 so as to enable communication between tracker electronics 140 and workstation 150. The connection between tracker electronics 140 and workstation 150 may also be a wireless or wired connection.

In operation, table sensors 125 and instrument sensor 135 may be embodied as wire coils useful in an electromagnetic tracking system. At least one of table sensors 125 and instrument sensor 135 generates (or transmits) at least one magnetic dipole field (not shown). By applying varying current to a wire coil in a sensor, a magnetic dipole field can be created. The magnetic dipole field may extend along any one of three orthogonal directions, as shown in FIG. 1 by the x-y-z Cartesian coordinate system. For example, one or several table sensors 125 may generate a magnetic dipole field along the y-direction.

Similarly, at least one of table sensors 125 and instrument sensor 135 may generate a plurality of magnetic dipole fields. When multiple coils are used in a sensor 125, 135 that is generating a magnetic field, each coil may be driven by the applied current at a different frequency to create dipole magnetic fields of differing directions and frequencies, thus making the various fields easily distinguishable. For example, each table sensor 125 or instrument sensor 135 may include three concentric orthogonal wire coils that create three frequency distinct magnetic dipole fields along each of the x-, y- and z-directions, as in an ISCA arrangement. However, table sensors 125 and/or instrument sensor 135 may also include a greater or lesser number of coils for use in generating magnetic fields. For example, table sensors 125 may each include six coils for generating magnetic fields.

Once at least one magnetic dipole field has been generated by either table sensors 125 or instrument sensor 135, at least one of table sensors 125 and instrument sensor 135 then measures at least one vector component (not shown) of the magnetic dipole field. A vector component of a magnetic field may be measured, for example, by an amount of mutual inductance in one or more coils of a sensor. A sensor 125, 135 may have one or more coils. For example, in an ISCA arrangement, instrument sensor 135 may have three concentric orthogonal wire coils to measure vector components of magnetic fields generated by table sensors 125. However, a sensor 125, 135 measuring vector components of magnetic fields may have as few as a single coil or may have a greater number of coils.

A sensor 125, 135 that measures a vector component may do so for each magnetic dipole field direction. For example, if table sensors 125 generate magnetic dipole fields along each of the x-, y- and z-directions, then instrument sensor 135 may measure vector components of the magnetic fields along each of the x-, y- and z-directions. Conversely, if instrument sensor 135 generates magnetic dipole fields along each of the x-, y- and z-directions, then table sensors 125 may measure vector components of the magnetic fields along each of the x-, y- and z-directions.

Either table sensor array 120 or instrument sensor 135 may be employed to generate one or more magnetic dipole fields. In an embodiment, if one or more table sensors 125 of array 120 generate one or more fields, then table sensors 125 are transmitting sensors. Consequently, instrument sensor 135 then acts as a receiving sensor and measures one or more vector components of the field(s).

If many or all table sensors 125 generate one or more dipole fields, then a volume of magnetic fields may be generated that encompasses part, substantially all, or all of table 110. The instrument 130 may then be moved relative to table 110 and array 120 within the volume of magnetic fields (shown in FIG. 1 as direction D₁ as an example). As instrument 130 and instrument sensor 135 are moved through the volume of magnetic fields, instrument sensor 135 may measure one or more vector components.

Instrument sensor 135 then communicates the measured vector component(s) to tracker electronics 140. As described above, instrument sensor 135 may communicate the vector component(s) over a wired or wireless communication connection. Once tracker electronics 140 receive the measured vector component(s), tracker electronics 140 determine at least one of a position and an orientation of instrument sensor 135 relative to array 120. For example, table sensors 125 can include three concentrically positioned orthogonal coils generating three magnetic dipole fields along each one of the x-, y- and z-directions. Similarly, instrument sensor 135 may include three concentrically positioned orthogonal coils measuring three vector components of each of the magnetic dipole fields at a location of instrument sensor 135. In such a system 100, instrument sensor 135 can measure three vector components of each of the three magnetic dipole fields at a location of instrument sensor 135, for example. Instrument sensor 135 therefore may provide nine vector component measurements of a volume of magnetic fields generated by table sensors 125, for example. These vector component measurements may then be communicated to tracker electronics 140, as described above.

In another embodiment, if instrument sensor 135 generates one or more dipole fields, then instrument sensor 135 can be transmitting sensor. Consequently, table sensors 125 then act as a receiving sensors and measure one or more vector components of the field(s).

Depending on the size of magnetic dipole fields generated by instrument sensor 135, a volume of magnetic fields may be generated that encompasses part, substantially all, or all of table 110 and/or a volume of a patient. The instrument 130 may then be moved relative to table 110 and array 120 within the volume of magnetic fields (shown in FIG. 1 as direction D₁ as an example). As instrument 130 and instrument sensor 135 are moved through the volume of magnetic fields, one or more table sensors 125 may measure one or more vector components.

As described above, by embedding tracking technology into table 110, array 120 can become fixed with respect to table 110. In so fixing array 120, magnetic field distortions normally caused by table 110 may be corrected by creating a magnetic field map at the time table 110 is manufactured. In contrast, by not embedding the tracking technology into table 110, any field distortion caused by table 110 must either be accounted for by creating a distortion-free table or by mapping the magnetic field before each and every use.

Table sensors 125 may then communicate the measured vector component(s) to tracker electronics 140. As described above, table sensors 125 may communicate the vector component(s) over a wired or wireless communication connection. Once tracker electronics 140 receive the measured vector component(s), tracker electronics 140 determine at least one of a position and an orientation of instrument sensor 135 relative to array 120. For example, instrument sensor 135 can include three concentrically positioned orthogonal coils generating three magnetic dipole fields along each one of the x-, y- and z-directions. Similarly, table sensors 125 may each include three concentrically positioned orthogonal coils measuring three vector components of each of the magnetic dipole fields at a location of each table sensor 125. In such a system 100, each table sensor 125 may measure three vector components of each of the three magnetic dipole fields at a location of the table sensor 125, for example. Table sensors 125 therefore may each provide nine vector component measurements of a volume of magnetic fields generated by instrument sensor 135, for example. These vector component measurements may then be communicated to tracker electronics 140, as described above.

Once tracker electronics 140 employ the communicated vector component(s) to determine at least one of a position and orientation of instrument sensor 135 relative to array 120, tracker electronics 140 communicate the at least one of a position and orientation to workstation 150.

When workstation 150 receives the position and/or orientation measurement(s), workstation 150 may use the measurement(s) to calculate a location of medical instrument 130 in an imaged volume. For example, instrument sensor 135 can be attached to instrument 130 in a known position at a known distance from a tip of instrument 130. Once a position and/or orientation of instrument sensor 135 is measured, workstation 150 may calculate a position and/or orientation of an instrument 130 tip based on the known position of instrument sensor 135 on instrument 130. During surgical procedures, for example, an instrument sensor 135 attached to a reducing rod or drill bit may be used to determine and display a position and/or orientation of instrument 130 in a patient. If a volume of the patient is being or has been imaged, for example by x-ray imaging, a user of system 100 may track the location of instrument 130 in the patient.

As described above, workstation 150 may include an output device, such as a computer monitor for example, to display an image of instrument 130 in an imaged volume. Workstation 150 may also include an input device, such as a keyboard or mouse, to allow a user to manipulate or calibrate images or system 100. Workstation 150 may also include a memory for storing, for example, a look-up table employed to calibrate system 100. For example, due to variations in generated magnetic dipole fields caused by, among other things, metal objects, a robot may be employed to generate a look-up table used to correlate a measured vector component to an actual measured vector component. A robot can include a predefined structure whose physical configuration and electric and magnetic properties are known such that the robot may be used to accurately measure magnetic fields and to characterize coils in an electromagnetic tracking system.

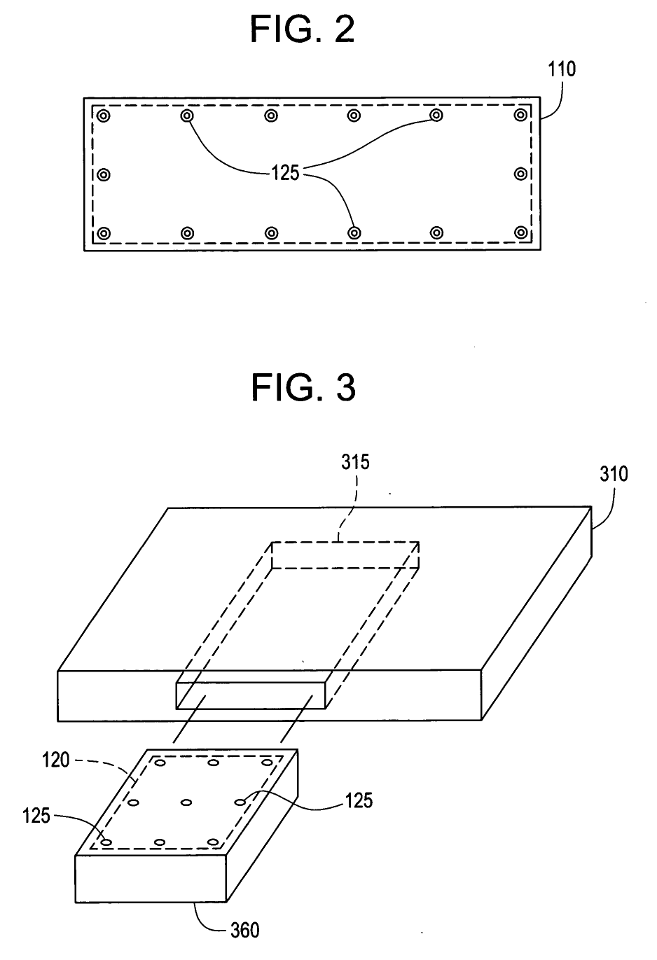

FIG. 2 illustrates medical table 110 used in accordance with an embodiment of the present invention. Table sensors 125 may be inserted along a periphery of table 110, as illustrated in FIG. 2. For example, table sensors 125 may be inserted along one or more edges of table 110 so that an area of table 110 is left without any regular pattern of table sensors 125.

FIG. 3 illustrates a retrofitted medical table 310 used in accordance with an embodiment of the present invention. Medical table 310 operates as described above with reference to table 110. Medical table 310 differs from table 110 in that table sensors 125 are attached or embedded in a form factor 360 that can be inserted into table slot 315. Form factor 360 may include an x-ray cartridge, for example, that is typically inserted into an x-ray cartridge slot (slot 315), for example. Once form factor 360 is inserted into slot 315, table sensors 125 may operate to generate magnetic fields or measure vector components of magnetic fields, as described above.

In another embodiment of the present invention, table sensors 125 and/or instrument sensor 135 may include one or more printed circuit board (“PCB”) multilayer coil or a stack or multiple layers of PCB coils.

In another embodiment of the present invention, table sensors 125 and/or instrument sensor 135 may include transceiver capabilities. For example, instrument sensor 135 or one or more table sensors 125 may include the capability to both transmit, or generate, one or more magnetic dipole fields and receive, or measure, one or more vector components of one or more magnetic fields. This capability allows for either array 120 or instrument sensor 135 to generate a volume of magnetic fields to be measured by an other sensor.

In another embodiment of the present invention, table sensors 125 and/or instrument sensor 135 may include transponder capabilities. Instrument sensor 135 or one or more table sensors 125 may include the capability to measure vector components of a magnetic dipole field and transmit this data to tracker electronics 140 only when the sensor is placed in the magnetic field. For example, if array 120 generates a volume of magnetic fields encompassing substantially all of an imaged volume and instrument sensor 135 is a transponder, then instrument sensor 135 does not communicate any measured vector components to tracker electronics 140 until instrument sensor 135 has been placed in the volume of magnetic fields and has measured at least one vector component.

In another embodiment of the present invention, tables 110, 310 may include additional extensions, such as arm, leg and/or head extensions to support extremities of a patient. Table sensors 125 may then be inserted in the additional extensions of tables 110, 310.

In another embodiment of the present invention, table sensors 125 may be embedded in the material of tables 110, 310. For example, table sensors 125 may be inserted into tables 110, 310 during its initial production and manufacture. Table sensors 125 may be embedded in radiolucent materials of tables 110, 310, such as fiberglass filled utlem, Kevlar or an other fibrous, non-conductive and non-magnetic material, for example.

In another embodiment of the present invention, tracker electronics 140 and workstation 150 may be integrally disposed in a single unit. For example, tracker electronics 140 may include a processor and associated software included in workstation 150.

In another embodiment of the present invention, tracker electronics 140 and/or workstation 150 may be integrally disposed in table 110, 310. Table 110, 310 and tracker electronics 140 and/or workstation 150 may therefore comprise a single physical unit, so as to reduce the amount of equipment in an operating or imaging room.

In another embodiment of the present invention, medical instrument 130 may be an object of interest. An object of interest may be any device or instrument that is to be tracked in a volume of a magnetic field. For example, an object of interest may be a surgical guide wire or a medical implant.

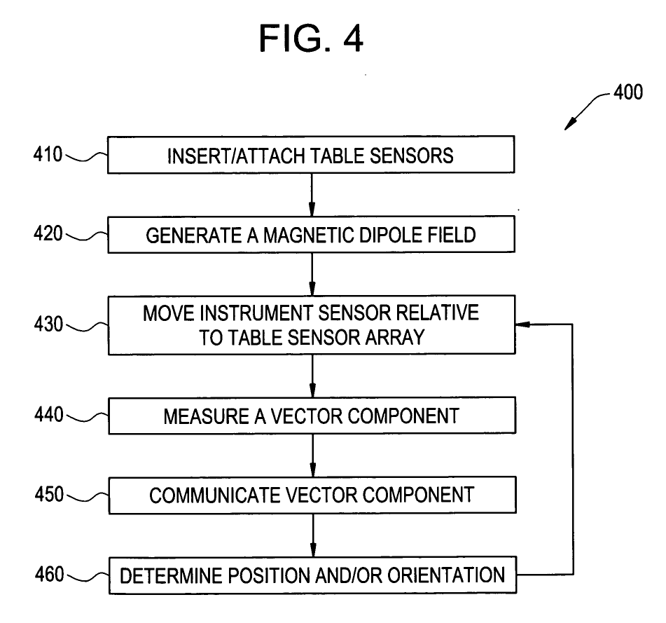

FIG. 4 illustrates a flow diagram for a method 400 for embedding tracking technology in a medical table used in accordance with an embodiment of the present invention. First, at step 410 a plurality of table sensors 125 are inserted or attached to a medical table 110, 310 to create sensor array 120. Next, at step 420, a magnetic dipole field can be generated by at least one of table sensors 125 and an instrument sensor 135 attached to an instrument 130. Several fields may be generated, as described above, by one or more sensors 125, 135.

Next, at step 430, instrument sensor 135 is moved relative to sensor array 120. At step 440, instrument sensor 135 measures at least one vector component of the one or more magnetic dipole fields generated at step 420. Multiple vector components may be measured at step 440.

Next, at step 450, instrument sensor 135 communicates the measured vector component(s) to tracking electronics 140. The communication may occur over a wired or wireless connection.

Next, at step 460, using the receiving vector component(s), tracking electronics 140 determine at least one of a position and an orientation of instrument sensor 135 relative to sensor array 120. After step 460, method 400 may proceed to step 430 if additional measurements are required or desired. For example, if method 400 is continuously tracking a position and/or orientation of instrument sensor 135, method 400 may continually loop among steps 430, 440, 450 and 460 to provide a continuous stream of measurements and determinations of a position and/or orientation of instrument sensor 135.

In another embodiment of the present invention, at step 410, table sensors 125 may be attached or inserted to a form factor 360 that is inserted into a table slot 315. For example, table sensors 125 may be attached or inserted in an x-ray cartridge or carrier 360 that is inserted into table slot 315.

In another embodiment of the present invention, at step 410, table sensors 125 may be embedded in the material of table 110, 310 during the construction of table 110, 310.

While particular elements, embodiments and applications of the present invention have been shown and described, it is understood that the invention is not limited thereto since modifications may be made by those skilled in the art, particularly in light of the foregoing teaching. It is therefore contemplated by the appended claims to cover such modifications and incorporate those features that come within the spirit and scope of the invention.

What is claimed is:

1. A medical table system with embedded tracking technology, said system including:
a plurality of table sensors inserted in said table to create a table sensor array;
an instrument sensor moved relative to said array;
at least one magnetic dipole field generated by at least one of said table sensors and said instrument sensor;
at least one vector component of said field measured by at least one of said table sensors and said instrument sensor; and
tracker electronics determining at least one of a position and an orientation of said instrument sensor relative to said array using said vector component.

2. The system of claim 1, wherein said array and said instrument sensor include at least one of a plurality of wire coils and a plurality of printed circuit board multilayer coils for use in an electromagnetic tracking system.

3. The system of claim 1, wherein said array generates said at least one magnetic dipole field and said instrument sensor measures said at least one vector component.

4. The system of claim 1, wherein said instrument sensor generates said at least one magnetic dipole field and at least one of said plurality of table sensors measures said at least one vector component.

5. The system of claim 1, wherein said array is inserted into said table along a periphery of said table.

6. The system of claim 1, wherein said array is inserted into said table in a grid formation throughout said table.

7. The system of claim 1, further including a form factor inserted into said table, said table sensors attached to said form factor.

8. The system of claim 1, wherein said tracker electronics are integrally disposed with said table.

9. A method for embedding tracking technology in a medical table, said method including:
inserting a plurality of table sensors in said table to create a table sensor array;
generating at least one magnetic dipole field;
moving an instrument sensor relative to said array;
measuring at least one vector component of said field; and
determining at least one of a position and an orientation of said instrument sensor relative to said array using said vector component.

10. The method of claim 9, wherein said array and said instrument sensor include at least one of a plurality of wire coils and a plurality of printed circuit board multilayer coils for use in an electromagnetic tracking system.

11. The method of claim 9, wherein said array generates said at least one magnetic dipole field and said instrument sensor measures said at least one vector component.

12. The method of claim 9, wherein said instrument sensor generates said at least one magnetic dipole field and at least one of said plurality of table sensors measures said at least one vector component.

13. The method of claim 9, wherein said array is inserted into said table along a periphery of said table.

14. The method of claim 9, wherein said array is inserted into said table in a grid formation throughout said table.

15. The method of claim 9, wherein said plurality of table sensors are inserted in said table by attaching said sensors to a form factor inserted into said table.

16. The method of claim 9, further including receiving a signal from at least one of said plurality of sensors and said instrument sensor at tracker electronics, said signal based on said vector component and employed to determine at least one of said position and said orientation, said tracker electronics integrally disposed with said table.

17. A wireless embedded tracking system, said system including:
a wireless sensor transmitting at least one magnetic dipole field;
an array of receiving sensors measuring said at least one field;
a medical table embedded with said array, said wireless sensor moved relative to said table; and
tracking electronics determining at least one of a position and an orientation of said wireless sensor relative to said array.

18. The system of claim 17, wherein said wireless sensor is attached to an object of interest, said object of interest moved relative to said table.

19. The system of claim 17, wherein said table is at least one of an operating table, an x-ray imaging table, a combination table and a Jackson table.

20. The system of claim 17, wherein said array of sensors are embedded in radiolucent material of said table.
